(19) United States
(12) Patent Application Publication
(10) Pub. No.: US 2011/0028444 A1
(43) Pub. Date: Feb. 3, 2011

(54) PHARMACEUTICALLY ACCEPTABLE SALTS OF ANTI-INFECTION QUINOLONE COMPOUNDS

(76) Inventors: Mao Chen, Guangdong (CN); Shaoxuan Zhu, Guangdong (CN); Lizhen Zheng, Guangdong (CN); Xuebin Liu, Guangdong (CN); Yuping Wang, Guangdong (CN); Shuwen Xu, Guangdong (CN)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/936,088

(22) PCT Filed: Apr. 2, 2009
(86) PCT No.: PCT/CN2009/071136

§ 371 Date: Oct. 1, 2010

(30) Foreign Application Priority Data
Apr. 3, 2008 (CN) 200810027211.9

(51) Int. Cl. A61K 31/397; C07D 513/04; A61P 31/00

(52) U.S. Cl. 514/210.16; 544/375

(57) ABSTRACT

The invention discloses the pharmaceutically acceptable salts of an optically active quinolone compound useful against infection, specifically the pharmaceutically acceptable salts of (S)-6-fluoro-1-methyl-4-oxo-7-(1-piperazinyl)-1H,4H-[1,3]thiazeto[3,2-a]quinoline-3-carboxylic acid. The pharmaceutically acceptable salts of the present invention are stable and have improved water solubility. They possess higher biological activity, less toxicity for kidney and no irritation to skin and muscle.

### FIELD OF THE INVENTION

This application is based on Chinese Patent Application No. 200810027211.9; the disclosure of the Chinese Patent Application is incorporated herein by reference.

This invention relates to a fluorine-containing, optically active quinolone compound for anti-infection drugs. More particularly, the invention relates to a pharmaceutically acceptable salt formed by (S)-6-fluoro-1-methyl-4-oxo-7-(1-piperazinyl)-1H,4H-[1,3]thiazeto[3,2-a]quinoline-3-carboxylic acid and an organic or inorganic acid.

### BACKGROUND OF THE INVENTION

Infectious diseases are the most common diseases involving virtually all clinical specialties. It is also one of the most common causes of death of patients. According to the report of the World Health Organization in 2000, the number of deaths caused by infectious diseases was 33.3% of the total number of deaths.

In the world market, the quinolone type drugs account for about 18% market share of the anti-infection drugs with an average annual growth rate of 7%. It continues to grow rapidly. Its total sales are second only to β-lactam drugs. Fluoroquinolones antibacterial drugs are also developing rapidly in recent years because of their relatively broader antibacterial spectrum and antibacterial activity. Both oral and injectable formulations of fluoroquinolones antibacterial drugs are commonly used in clinical applications in China. The antibacterial mechanism of fluoroquinolone is targeting the DNA of the bacteria, blocking the bacterial DNA topoisomerase from forming super-helical DNA, leading to irreversible damage to chromosomes, and preventing bacterial cell from division and breeding.

6-fluoro-1-methyl-4-oxo-7-(1-piperazinyl)-1H,4H-[1,3]thiazeto[3,2-a]quinoline-3-carboxylic acid is commonly called ulifloxacin internationally and “You Li Sha Xing” in Chinese, code NM394. Chinese invention patent CN101003540A disclosed the use of 6-fluoro-1-methyl-4-oxo-7-(1-piperazinyl)-1H,4H-[1,3]thiazeto[3,2-a]quinoline-3-carboxylic acid mesylate in the preparation of anti-infection drugs. The disclosed compounds were racemic isomers that contained the same amount of S and R structures of ulifoxacin salts; their stero configurations can be expressed as (±).

Kise Masahiro et al., Japanese Patent Application Publication No. 3-218383, disclosed the use of laboratory HPLC with 3×50 mm of ODS column to separate racemic 6-fluoro-1-methyl-4-oxo-7-(1-piperazinyl)-1H,4H-[1,3]thiazeto[3,2-a]quinoline-3-carboxylic acid and about 80 mg of S and R structures were obtained, defined as L- and R-isomers. However, the disclosed separation method has limited capacity and high cost, and thus is not suitable for commercial production. L-isomers of the chemical, called (S)-6-fluoro-1-methyl-4-oxo-7-(1-piperazinyl)-1H,4H-[1,3]thiazeto[3,2-a]quinoline-3-carboxylic acid (or L-You Li Sha Xing), structure type 2, see below.

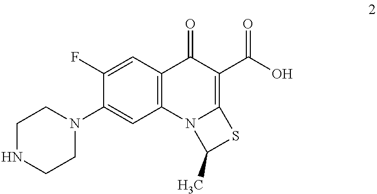

Jun Segawa, et al., Chem Pharm Bull (Tokyo), 1995 July; 43 (7): 1238, reported that they tested the in-vitro antibacterial activity of (S)-6-fluoro-1-methyl-4-oxo-7-(1-piperazinyl)-1H,4H-[1,3]thiazeto[3,2-a]quinoline-3-carboxylic acid and (R)-6-fluoro-1-methyl-4-oxo-7-(1-piperazinyl)-1H,4H-[1,3]thiazeto[3,2-a]quinoline-3-carboxylic acid showing that the activity of the S isomer was 2 to 8 times more potent that of the R isomer. However, the S isomer had low water solubility, which limited its clinical applications.

Although racemic ulifloxacin has an excellent antibacterial activity, it also has relatively high toxicity. Ishida S, Journal of Toxicological Sciences, 1996 June, 21 Suppl 1:131, reported the toxicity of racemic ulifloxacin NM394 to rats by intravenous injection. Male and female groups of Sprague-Drwlog rats were, respectively, intravenously injected with 3, 10 and 30 mg/kg doses of NM394. For a period of four weeks, the rats injected with 10 and 30 mg/kg doses had shown significant increase in water consumption and urinary amount. Crystalline substance and epithelial cells were found in urinary precipitation. For the group of rats injected with 30 mg/kg dose, their urea became cloudy. The rats injected with 10 and 30 mg/kg doses showed reduced serum γ-globulin. The groups of mice injected with 10 and 30 mg/kg doses had increased blood urea nitrogen and creatinine, indicating their kidney function was damaged. In addition, the rats injected with 10 and 30 mg/kg doses showed pathological changes such as tubular nephropathy, and crystalline material was found. The rats injected with 30 mg/kg dose showed increased weights of kidney and appendix. The 3 mg/kg dose group did not show significant problems; thus for rats, the NOAEL (no observed adverse effect level) dose of NM394 should be 3 mg/kg.

Shimazu H et al., Journal of Toxicological Sciences, 1996, June, 21 Suppl 1:33, reported the intravenous injection of NM394 caused rats to suffer from convulsions, difficult breathing, injection site redness, swelling, and necrosis, and caused mice to suffer from losing weight, testicular atrophy, and visible pulmonary congestion.

The above references indicate that racemic ulifloxacin has high irritation and toxicity, and thus it is of no clinical significance.

### DESCRIPTION OF THE INVENTION

The objective of the invention is to provide an optically active quinolone compound which can be used as an anti-infection medicine. The compound is readily soluble in water and has a higher antibacterial activity than NM394. It has low toxicity and low irritation to muscle and skin. It has low side effects, broad antibacterial spectrum, and is more suitable for clinic uses.

The technical scheme of the invention is to provide an anti-infection drug, which is an optically active quinolone compound having the following general structure:

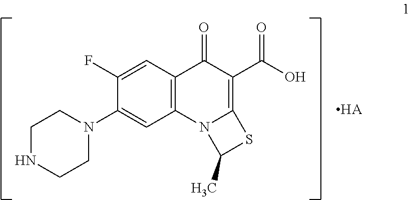

In the above structure, HA is an organic or inorganic acid which forms a pharmaceutically acceptable compound with (S)-6-fluoro-1-methyl-4-oxo-7-(1-piperazinyl)-1H,4H-[1,3]thiazeto[3,2-a]quinoline-3-carboxylic acid.

In particular, S-ulifoxacin is isolated from racemic ulifloxacin, which then forms a pharmaceutically acceptable compound with an organic or inorganic acid.

Commonly used organic acids include acetic acid, glycine, methylsulfonic acid, lactic acid, glutamic acid, mandelic acid, gluconic acid, aspartic acid, citric acid, succinic acid, fumaric acid, maleic acid, oxalic acid, lactose acid, and benzenesulfonic acid; and the inorganic acids include hydrochloric acid, hydrobromic acid, sulfuric acid, and phosphoric acid. Suitable aspartic acid includes its DL, D, and L forms. Preferred acids include methylsulfonic acid, lactic acid, gluconic acid, gluconic acid, and aspartic acid.

The product formed by lactic acid and (S)-6-fluoro-1-methyl-4-oxo-7-(1-piperazinyl)-1H,4H-[1,3]thiazeto[3,2-a]quinoline-3-carboxylic acid is called (S)-6-fluoro-1-methyl-4-oxo-7-(1-piperazinyl)-1H,4H-[1,3]thiazeto[3,2-a]quinoline-3-carboxylic acid lactate. The product has rotation [α]_{D}²⁰ from −112.5 to −118.2°; IR 1698 cm⁻¹, 1629 cm⁻¹, 1605 cm⁻¹, 1501 cm⁻¹, 1396 cm⁻¹, and 1257 cm⁻¹.

The product formed by methylsulfonic acid and (S)-6-fluoro-1-methyl-4-oxo-7-(1-piperazinyl)-1H,4H-[1,3]thiazeto[3,2-a]quinoline-3-carboxylic acid is called (S)-6-fluoro-1-methyl-4-oxo-7-(1-piperazinyl)-1H,4H-[1,3]thiazeto[3,2-a]quinoline-3-carboxylic acid mesylate. The product has rotation [α]_{D}²⁰ from −106.6 to −112.6°; IR 1707 cm⁻¹, 1629 cm⁻¹, 1602 cm⁻¹, and 1501 cm⁻¹.

The product formed by gluconic acid and (S)-6-fluoro-1-methyl-4-oxo-7-(1-piperazinyl)-1H,4H-[1,3]thiazeto[3,2-a]quinoline-3-carboxylic acid is called (S)-6-fluoro-1-methyl-4-oxo-7-(1-piperazinyl)-1H,4H-[1,3]thiazeto[3,2-a]quinoline-3-carboxylic acid gluconate. The product has IR 1695 cm⁻¹, 1629 cm⁻¹, 1601 cm⁻¹, and 1503 cm⁻¹.

The product formed by glutamic acid and (S)-6-fluoro-1-methyl-4-oxo-7-(1-piperazinyl)-1H,4H-[1,3]thiazeto[3,2-a]quinoline-3-carboxylic acid is called (S)-6-fluoro-1-methyl-4-oxo-7-(1-piperazinyl)-1H,4H-[1,3]thiazeto[3,2-a]quinoline-3-carboxylic acid glutamate. The product has IR 1628 cm⁻¹, 1603 cm⁻¹, 1499 cm⁻¹, 1457 cm⁻¹, 1397 cm⁻¹, 1257 cm⁻¹.

The product formed by aspartic acid and (S)-6-fluoro-1-methyl-4-oxo-7-(1-piperazinyl)-1H,4H-[1,3]thiazeto[3,2-a]quinoline-3-carboxylic acid is called (S)-6-fluoro-1-methyl-4-oxo-7-(1-piperazinyl)-1H,4H-[1,3]thiazeto[3,2-a]quinoline-3-carboxylic acid aspartate. The product has IR 1695 cm⁻¹, 1628 cm⁻¹, 1602 cm⁻¹, and 1499 cm⁻¹.

According to the invention, the compound 1 is obtained by the following method. Reacting D-tartrate solution in DMSO with (±)-6-fluoro-1-methyl-4-oxo-7-(1-piperazinyl)-1H,4H-[1,3]thiazeto[3,2-a]quinoline-3-carboxylic acid solution in DMSO yields a tartrate salt precipitate. Hydrolysis of the tartrate salt with NaOH solution yields (S)-6-fluoro-1-methyl-4-oxo-7-(1-piperazinyl)-1H,4H-[1,3]thiazeto[3,2-a]quinoline-3-carboxylic acid.

At 0 to 60° C., an HA solution having a given concentration was prepared; to the solution was then added (S)-6-fluoro-1-methyl-4-oxo-7-(1-piperazinyl)-1H,4H-[1,3]thiazeto[3,2-a]quinoline-3-carboxylic acid under agitation. The reaction continues for 1 to 6 hours until the reactor contents become clear and no solid substance was observed. Active carbon black (5%) was added to the reaction solution and the mixture was then filtered. An organic solvent (1 to 100 times) was added to precipitate the product. The product was filtered and dried to yield compound 1. The above-mentioned organic solvent was selected from methanol, ethanol, isopropyl alcohol, acetone, tetrahydrofuran, and a mixture thereof. The feed ratio of (S)-6-fluoro-1-methyl-4-oxo-7-(1-piperazinyl)-1H,4H-[1,3]thiazeto[3,2-a]quinoline-3-carboxylic acid to HA is 1:0.8 to 1.5. HA is any acid described above; according to this method a corresponding compound can be obtained.

The invention also provides anti-infection compositions which comprise the optical active, fluorine-containing quinolone compound 1 as an active ingredient and one or more pharmaceutically acceptable conventional carriers. For instance, the anti-infection composition comprises conventional excipients so that they can be converted to oral dosage forms such as medicinal tablets, capsules (including sustained-release and controlled-release formulations), powders, and granular agent of solid, or to non-gastrointestinal delivery forms, such as injection agent.

The Effectiveness of the Invention:

The compound of the invention has stable properties. Compared to ulifloxacin, it has improved water solubility and reduced pH value in an aqueous solution. It is readily soluble in water; it has high antibacterial activity, low nephrotoxicity, and no irritation to muscle and skin. In the experiments, the level of no adverse drug effect is 30 mg/kg, which is 10 times higher than that of ulifloxacin. It has low side effects, broad antibacterial spectrum, and its activity is 1-3 times higher than the DL-ulifloxacin.

The following examples further illustrate the technical scheme and effectiveness of the invention. However, these examples do not limit the scope of the invention.

### Example 1

### Preparation of (S)-6-fluoro-1-methyl-4-oxo-7-(1-piperazinyl)-1H,4H-[1,3]thiazeto[3,2-a]quinoline-3-carboxylic acid

Racemic ulifloxacin (105 g) was dissolved in DMSO (1500 mL). D-tartrate (27 g) solution in DMSO (405 mL) was added to the racemic ulifoxacin solution with agitation. Cloudiness and precipitation appeared. After 20 hours of agitation at an ambient temperature, the mixture was filtered. The solid was dried under vacuum to yield 86 g of solid. The solid was recrystallized in DMSO to yield 37 g of (S)-6-fluoro-1-methyl-4-oxo-7-(1-piperazinyl)-1H,4H-[1,3]thiazeto[3,2-a]quinoline-3-carboxylic acid-D-tartrate salt; elemental analysis indicated: C 49.08%, H 5.06%, N 9.50%, and S 7.44% (corresponding to: C₁₆H₁₆FN₃O₃S.1/2C₄H₆O₆.H₂O, calculated value: C 48.86%, H 4.78, N 9.50%, and S 7.25%). The salt was dispersed in water and the dispersion was neutralized with 2% NaOH aqueous solution to a pH value of 7 to 8. The precipitate was filtered and dried to yield 24.5 g of (S)-6-fluoro-1-methyl-4-oxo-7-(1-piperazinyl)-1H,4H-[1,3]thiazeto[3,2-a]quinoline-3-carboxylic acid. It had a rotation [α]_{D}²⁰=−143.4° (c=0.15, 0.1 mol/L NaOH), ¹H-NMR (DMSO-d6) δ2.11 (3H, d, j=6.2 Hz), 2.85-3.20 (8H, m), 6.40 (1H, q, j=6.2 Hz), 6.89 (1H, d, j=7.4 Hz), 7.79 (1H, d, j=13.9 Hz), optical purity e.e.>95%.

### Example 2

### Preparation of (R)-6-fluoro-1-methyl-4-oxo-7-(1-piperazinyl)-1H,4H-[1,3]thiazeto[3,2-a]quinoline-3-carboxylic acid (Compound 3)

Racemic ulifloxacin (105 g) was dissolved in DMSO (1500 mL). L-tartrate (27 g) solution in DMSO (405 mL) was added to the racemic ulifoxacin solution with agitation. Cloudiness and precipitation appeared. After 20 hours of agitation at an ambient temperature, the mixture was filtered and dried under vacuum to yield 82 g of solid. The solid was recrystallized in DMSO to yield 34 g of (R)-6-fluoro-1-methyl-4-oxo-7-(1-piperazinyl)-1H,4H-[1,3]thiazeto[3,2-a]quinoline-3-carboxylic acid-L-tartrate salt. The salt was dispersed in water and the dispersion was neutralized with 2% NaOH aqueous solution to a pH value of 7 to 8. The precipitate was filtered and dried to yield 22 g of (R)-6-fluoro-1-methyl-4-oxo-7-(1-piperazinyl)-1H,4H-[1,3]thiazeto[3,2-a]quinoline-3-carboxylic acid. It had specific rotation [α]_{D}²⁰=+139.2° (c=0.15, 0.1 mol/L NaOH), optical purity e.e>95%.

### Example 3

### Preparation of (S)-6-fluoro-1-methyl-4-oxo-7-(1-piperazinyl)-1H,4H-[1,3]thiazeto[3,2-a]quinoline-3-carboxylic acid lactate (Compound 4)

At 20° C., a flask was charged with 30 mL of water, 1.6 g of lactic acid, and then with 5 g of (S)-6-fluoro-1-methyl-4-oxo-7-(1-piperazinyl)-1H,4H-[1,3]thiazeto[3,2-a]quinoline-3-carboxyl is acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol to precipitate the solid. After 2 hours of agitation, the mixture was filtered; the solid cake was crushed and dried at 50° C. under vacuum to yield 3.6 g of (S)-6-fluoro-1-methyl-4-oxo-7-(1-piperazinyl)-1H,4H-[1,3]thiazeto[3,2-a]quinoline-3-carboxylic acid lactate. [α]_{D}²⁰=−116.5 (c=1.0, H₂O); ¹H-NMR (D₂O) δ1.30 (3H, d, j=6.4 Hz), 2.06 (3H, d, j=6.0 Hz), 3.44 to 3.51 (8H, m), 4.10 (1H, q, j=10.4 Hz), 6.07 (1H, q, j=6.4 Hz), 6.47 (1H, d, j=6.8 Hz), 7.24 (1H, d, j=13.21 Hz), IR 1698 cm⁻¹, 1629 cm⁻, 1605 cm⁻¹, 1501 cm⁻, 1396 cm⁻, and 1257 cm⁻¹.

### Example 4

### Preparation of (S)-6-fluoro-1-methyl-4-oxo-7-(1-piperazinyl)-1H,4H-[1,3]thiazeto[3,2-a]quinoline-3-carboxylic acid mesylate (Compound 5)

At 20° C., a flask was charged with 30 mL of water, 2.1 g of methylsulfonic acid, and then with 5 g of (S)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 60° C. under vacuum to yield 4.0 g of (S)-6-fluoro-1-methyl-4-oxo-7-(1-piperazinyl)-1H,4H-[1,3]thiazeto[3,2-a]quinoline-3-carboxylic acid mesylate. [α]_{D}²⁰=−109.4° (c=1.0, H₂O), IR 1707 cm⁻¹, 1629 cm⁻¹, 1602 cm⁻, 1501 cm⁻¹; ¹H-NMR (D₂O) δ2.00 (3H, d, j=6 Hz), 2.73 (3H.s), 3.39-3.45 (8H, m), 6.05 (1H, q, j=6 Hz), 6.42 (1H, d, j=5.2 Hz), 7.21 (1H, d, j=13.2 Hz); elemental analysis: C 45.32%, H 4.85%, N 9.25%, (C₁₆H₁₆FN₃O₃S.CH₄O₃S.1/2H₂O; calculated value: C 44.93%, H 4.66% and N 9.25%).

### Example 5

### Preparation of (S)-6-fluoro-1-methyl-4-oxo-7-(1-piperazinyl)-1H,4H-[1,3]thiazeto[3,2-a]quinoline-3-carboxylic acid gluconate (Compound 6)

At 20° C., a flask was charged with 30 mL of water, 6.8 mL of 50% gluconic acid aqueous solution, and then with 5 g of (S)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 50° C. under vacuum to yield 4.0 g of (S)-6-fluoro-1-methyl-4-oxo-7-(1-piperazinyl)-1H,4H-[1,3]thiazeto[3,2-a]quinoline-3-carboxylic acid gluconate.

Analysis results: IR 1695 cm⁻¹, 1629 cm⁻¹, 1601 cm⁻¹, 1503 cm⁻¹; ¹H-NMR (DMSO-d₆) 62.12 (3H, s), 6.39 (1H, s), 6.95 (1H, s), and 7.80 (1H, d, j=13.6 Hz).

### Example 6

### Preparation of (S)-6-fluoro-1-methyl-4-oxo-7-(1-piperazinyl)-1H,4H-[1,3]thiazeto[3,2-a]quinoline-3-carboxylic acid aspartate (Compound 7)

At 20° C., a flask was charged with 30 mL of water, 20 g of aspartic acid, and then with 5 g of (S)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 50° C. under vacuum to yield 3.7 g of (S)-6-fluoro-1-methyl-4-oxo-7-(1-piperazinyl)-1H,4H-[1,3]thiazeto[3,2-a]quinoline-3-carboxylic acid asparate. IR 1695 cm⁻¹, 1628 cm⁻¹, 1602 cm⁻¹, 1499 cm⁻¹; ¹H-NMR (D₂O) δ2.13 (2H, m), 2.70-2.89 (3H, s), 3.50˜3.57 (8H, m), 3.93 (1H, q, j=4.8 Hz), 6.16 (1H, s), 6.57 (1H, s), and 7.35 (1H, d, j=12.8 Hz).

### Example 7

### Preparation of (S)-6-fluoro-1-methyl-4-oxo-7-(1-piperazinyl)-1H,4H-[1,3]thiazeto[3,2-a]quinoline-3-carboxylic acid glutamate (Compound 8)

At 20° C., a flask was charged with 30 mL of water, 2.8 mL g of glutamic acid, and then with 5 g of (S)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 50° C. under vacuum to yield 3.6 g of (S)-6-fluoro-1-methyl-4-oxo-7-(1-piperazinyl)-1H,4H-[1,3]thiazeto[3,2-a]quinoline-3-carboxylic acid glutamate. ¹H-NMR (D₂O) δ2.1˜2.5 (6H, m), 2.40˜2.47 (3H, m), 3.49˜3.59 (8H, m), 3.80 (1H, q, j=1.6 Hz), 6.09 (1H, s), 6.49 (1H, s), and 7.20 (1H, d, j=13.2 Hz).

### Example 8

### Preparation of (R)-6-fluoro-1-methyl-4-oxo-7-(1-piperazinyl)-1H,4H-[1,3]thiazeto[3,2-a]quinoline-3-carboxylic acid mesylate (Compound 9)

At 20° C., a flask was charged with 30 mL of water, 2.1 mL g of methylsulfonic acid, and then with 5 g of (R)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 60° C. under vacuum to yield 4.0 g of (R)-6-fluoro-1-methyl-4-oxo-7-(1-piperazinyl)-1H,4H-[1,3]thiazeto[3,2-a]quinoline-3-carboxylic acid mesylate. [α]_{D}²⁰=+107.1°, (c=1.0, H₂O), IR 1705 cm⁻¹, 1628 cm⁻¹, 1602 cm⁻¹, 1501 cm⁻¹; ¹H-NMR (D₂O) δ2.02 (3H, d, j=3.6 Hz), 2.73 (3H, s), 3.40-3.48 (8H, m), 6.09 (1H, s), 6.46 (1H, d, j=5.2 Hz), 7.21 (1H, d, j=13.2 Hz); elemental analysis C 44.60%, H 5.01%, N 9.08%, (C₁₆H₁₆FN₃O₃S.CH₄O₃S.1/2H₂O; calculated C 44.93%, H 4.66% and N 9.25%).

### Example 9

### Preparation of (R)-6-fluoro-1-methyl-4-oxo-7-(1-piperazinyl)-1H,4H-[1,3]thiazeto[3,2-a]quinoline-3-carboxylic acid gluconate (Compound 10)

At 20° C., a flask was charged with 30 mL of water, 6.8 mL g of 50% gluconic acid aqueous solution, and then with 5 g of (R)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 50° C. under vacuum to yield 4.0 g of (R)-6-fluoro-1-methyl-4-oxo-7-(1-piperazinyl)-1H,4H-[1,3]thiazeto[3,2-a]quinoline-3-carboxylic acid.

### Example 10

### Preparation of (R)-6-fluoro-1-methyl-4-oxo-7-(1-piperazinyl)-1H,4H-[1,3]thiazeto[3,2-a]quinoline-3-carboxylic acid aspartate (Compound 11)

At 20° C., a flask was charged with 30 mL of water, 20 g of aspartic acid, and then with 5 g of (R)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 60° C. under vacuum to yield 3.7 g of (R)-6-fluoro-1-methyl-4-oxo-7-(1-piperazinyl)-1H,4H-[1,3]thiazeto[3,2-a]quinoline-3-carboxylic acid aspirate.

### Example 11

### Preparation of (R)-6-fluoro-1-methyl-4-oxo-7-(1-piperazinyl)-1H,4H-[1,3]thiazeto[3,2-a]quinoline-3-carboxylic acid glutamate (Compound 12)

At 20° C., a flask was charged with 30 mL of water, 2.8 mL of glutamic acid, and then with 5 g of (R)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid was dried at 50° C. under vacuum to yield 3.6 g of (R)-6-fluoro-1-methyl-4-oxo-7-(1-piperazinyl)-1H,4H-[1,3]thiazeto[3,2-a]quinoline-3-carboxylic acid glutamate.

### Example 12

### Preparation of (R)-6-fluoro-1-methyl-4-oxo-7-(1-piperazinyl)-1H,4H-[1,3]thiazeto[3,2-a]quinoline-3-carboxylic acid lactate (Compound 13)

At 20° C., a flask was charged with 30 mL of water, 1.6 g of lactic acid, and then with 5 g of (R)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was added 200 mL of anhydrous ethanol under agitation for 1 hour to precipitate the solid. After additional 2 hours of agitation, the mixture was filtered; the solid cake was crushed and dried at 50° C. under vacuum to yield 3.6 g of (R)-6-fluoro-1-methyl-4-oxo-7-(1-piperazinyl)-1H,4H-[1,3]thiazeto[3,2-a]quinoline-3-carboxylic acid lactate.

### Example 13

### Preparation of (S)-6-fluoro-1-methyl-4-oxo-7-(1-piperazinyl)-1H,4H-[1,3]thiazeto[3,2-a]quinoline-3-carboxylic acid chloride

At 20° C., a flask was charged with 30 mL of water, 1.6 g of lactic acid, and then with 5 g of (S)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was dropwise added 1.8 mL of 36% hydrochloric acid under agitation to precipitate the solid. After additional 2 hours of agitation, the mixture was filtered; the solid cake was crushed, washed with water and dried at 50° C. under vacuum to yield 4.6 g of (S)-6-fluoro-1-methyl-4-oxo-7-(1-piperazinyl)-1H,4H-[1,3]thiazeto[3,2-a]quinoline-3-carboxylic acid chloride.

### Example 14

### Preparation of (S)-6-fluoro-1-methyl-4-oxo-7-(1-piperazinyl)-1H,4H-[1,3]thiazeto[3,2-a]quinoline-3-carboxylic acid sulfate

At 20° C., a flask was charged with 30 mL of water, 1.6 g of lactic acid, and then with 5 g of (S)-6-fluoro-1-methyl-4-oxo-7-(1-piperazinyl)-1H,4H-[1,3]thiazeto[3,2-a]quinoline-3-carboxylic acid under agitation. After 60 minutes of agitation, an essentially clear liquid was obtained. To the liquid was added 5% of active carbon to discolor for 30 minutes. The mixture was filtered. To the filtrate was dropwise added 1.4 mL of 98% sulfuric acid under agitation to precipitate the solid. After additional 2 hours of agitation, the mixture was filtered; the solid cake was crushed, washed with water, and dried at 50° C. under vacuum to yield 4.8 g of (S)-6-fluoro-1-methyl-4-oxo-7-(1-piperazinyl)-1H,4H-[1,3]thiazeto[3,2-a]quinoline-3-carboxylic acid sulfate.

### Example 15

### Solubility Test

Three lots of mesylates and three lots of lactates, respectively, were subjected to preliminary tests for maximum solubility in water. At ambient temperature (25° C.), 0.2 g of sample was dissolved in 1 mL of water. To the solution was added 0.1 g of the sample each time until the sample could no longer be dissolved. The amount of sample dissolved was the maximum solubility. The value was calculated by the average of two tests; the results are listed in the following table.

| | | | Water |
|---|---|---|---|
| | | | Solubility |
| Isomers | Compounds | | mg/mL |
| Racemic (±) | Mesylate | Prepared according to | 340 |
| | | Chinese Pat. Appl. No. | |
| | | 200610033028.0. | |
| | Lactate | Prepared according to | 106 |
| | | Chinese Pat. Appl. No. | |
| | | 200610033042.0. | |
| Levorotary | Mesylate | Compound 5 of Example 4 | 490 |
| (S) | Lactate | Compound 4 of Example 3 | 70 |
| Dextrorotatory | Mesylate | Compound 9 of Example 8 | 501 |
| (L) | Lactate | Compound 13 of Example | 116 |
| | | 12 | |

The results show that the mesylates have better water solubility than lactates, and levorotary mesylates have better solubility than racemic mesylates.

### Example 16

### In-Vitro Antibacterial Tests

Test method: the agar dilution method was used for the minimum inhibitory concentration (MIC) determination. The antibacterial chemicals were mixed with given amounts of agar in various concentrations and the mixtures were made into solid plates. Each plate had the antibacterial concentration two times higher than the next plate. Bacteria species were then added to the surface of the plates, cultured, and observed for their growth. According to the U.S. Committee for Clinical Laboratory Standards (NCCIS) standard, the test plates were placed in dark, non-reflective surfaces to determine the end point of the bacteria growth; the lowest concentration at which no bacteria growth was observed was reported as the MIC. In all the experiments, the MIC of the quality control strains met the quality control standards of the NCCIS.

If the bacteria species grew on the tested plates which had the concentration higher than the end point, or if the bacteria species grew on a plate which had a higher concentration but did not grow on the plate which had a lower concentration, the purity of the cultures would be double-checked or the tests were repeated.

Test Samples:

Ciprofloxacin, levofloxacin, and (±) ulifloxacin from the market were used as controls.
(S)-6-fluoro-1-methyl-4-oxo-7-(1-piperazinyl)-1H,4H-[1,3]thiazeto[3,2-a]quinoline-3-carboxylic acid mesylate (Compound 5);
(S)-6-fluoro-1-methyl-4-oxo-7-(1-piperazinyl)-1H,4H-[1,3]thiazeto[3,2-a]quinoline-3-carboxylic acid lactate (Compound 4);
(S)-6-fluoro-1-methyl-4-oxo-7-(1-piperazinyl)-1H,4H-[1,3]thiazeto[3,2-a]quinoline-3-carboxylic acid glutamate (Compound 8);
(S)-6-fluoro-1-methyl-4-oxo-7-(1-piperazinyl)-1H,4H-[1,3]thiazeto[3,2-a]quinoline-3-carboxylic acid gluconate (Compound 6); and
(S)-6-fluoro-1-methyl-4-oxo-7-(1-piperazinyl)-1H,4H-[1,3]thiazeto[3,2-a]quinoline-3-carboxylic acid aspartate (Compound 7) are prepared according to the examples of this invention.

(±)-6-fluoro-1-methyl-4-oxo-7-(1-piperazinyl)-1H,4H-[1,3]thiazeto[3,2-a]quinoline-3-carboxylic acid lactate was prepared according to the examples of Chinese Pat. Appl. No. 200610033042.0.

(±)-6-fluoro-1-methyl-4-oxo-7-(1-piperazinyl)-1H,4H-[1,3]thiazeto[3,2-a]quinoline-3-carboxylic acid mesylate was prepared according to the examples of Chinese Pat. Appl. No. 200610033028.0.

(±)-6-fluoro-1-methyl-4-oxo-7-(1-piperazinyl)-1H,4H-[1,3]thiazeto[3,2-a]quinoline-3-carboxylic acid gluconate was prepared according to the examples of Chinese Pat. Appl. No. 200610033033.1.

(±)-6-fluoro-1-methyl-4-oxo-7-(1-piperazinyl)-1H,4H-[1,3]thiazeto[3,2-a]quino line-3-carboxylic acid aspartate was prepared according to the examples of Chinese Pat. Appl. No. 200610033034.6.

(±)-6-fluoro-1-methyl-4-oxo-7-(1-piperazinyl)-1H,4H-[1,3]thiazeto[3,2-a]quinoline-3-carboxylic acid glutamate was prepared according to the examples of Chinese Pat. Appl. No. 200610033039.9.

The test bacterial strains were the standard quality control test strains of Klebsiella pneumoniae strains (Strains No. CMCC 46114-8), Pseudomonas aeruginosa (Strains No. ATCC 27853), Escherichia coli (Strains No. ATCC25922), and Staphylococcus aureus cocci (Strains No. ATCC25925). Other bacteria included those isolated from sputum, throat swab or urine of patients with acute bacterial respiratory tract infection or urinary tract infection and identified by the hospitals.

(S)-6-fluoro-1-methyl-4-oxo-7-(1-piperazinyl)-1H,4H-[1,3]thiazeto[3,2-a]quino line-3-carboxylic acid lactate and other test samples, equivalent to 96 mg of ulifloxacin, were dissolved in sterilized distilled water to make up 50 mL of each solution. HCl levofloxacin injection and NaCl ciprofloxacin lactate injection were diluted with sterilized distilled water to a concentration of 1920 μg/mL. The in-vitro antibacterial test results are listed in Table 1.

TABLE 1
In-vitro MIC (μg/mL)

| | | Bacteria (ested | | | | | | | |
|---|---|---|---|---|---|---|---|---|---|
| | Stero- | Pseudomonas | Staphylococcus | Enterococcus | Streptococcus | Escherichia | Klebsiella | Enterobacter | Vibrio |
| Sample Name | structure | aeruginosa | aureus | faecalis | pneumoniae | coli | pneumoniae | cloacae | cholerae |
| Levofloxacin | (S) | 2 | 0.5 | — | — | 0.0313 | 0.0125 | — | — |
| Ciprofloxacin | (±) | 0.5 | 0.5~1 | — | — | 0.0313 | 0.0313~0.0625 | — | — |
| Compound 2 | (S) | 0.177 | 0.177 | — | 0.025 | 0.0125 | | 0.5 | — |
| Compound 3 | (R) | 2 | 2.828 | — | 0.25 | 0.225 | — | 4 | — |
| Ulifloxacin | (±) | 0.5 | 0.5 | — | 0.031 | 0.08 | — | 1 | — |
| Ulifloxaci | (S) | 0.25 | 0.125 | 0.5 | 0.02 | 0.01 | — | 0.5 | — |
| lactate | (R) | 1 | 1 | 4 | 0.125 | 0.25 | — | 4 | — |
| | (±) | 0.5 | 0.25 | 2 | 0.1 | 0.125 | — | 1.25 | — |
| Ulifloxaci | (S) | 0.25 | 0.25 | 0.2 | 0.01 | 0.0125 | 0.02 | 0.5 | 0.02 |
| mesylate | (R) | 1 | 2 | 4 | 0.125 | 0.225 | 0.5 | 4 | 2 |
| | (±) | 0.5 | 0.5 | 1.5 | 0.088 | 0.063 | 0.2 | 1 | 0.5 |
| Ulifloxaci | (S) | 0.25 | 0.25 | — | — | 0.01 | 0.02 | — | 0.02 |
| gluconate | (R) | 1 | 1 | — | — | 0.25 | 0.5 | — | 2 |
| | (±) | 0.5 | 0.5 | — | — | 0.05 | 0.2 | — | 0.5 |
| Ulifloxaci | (S) | 0.25 | 0.25 | — | — | 0.02 | — | — | — |
| aspartate | (R) | 1 | 2 | — | — | 0.25 | — | — | — |
| | (±) | 0.5 | 0.5 | — | — | 0.1 | — | — | — |
| Ulifloxaci | (S) | — | 0.25 | — | 0.125 | 0.01 | — | — | — |
| glutamate | (R) | — | 2 | — | 0.5 | 0.5 | — | — | — |
| | (±) | — | 0.5 | — | 0.2 | 0.125 | — | — | — |

The results indicate that the antibacterial activity of the S-ulifloxacin is 3 to 10 times of the R-ulifloxacin and two or more times of racemic ulifloxacin. Compounds 2, 4, 5, 6, 7, and 8 have relatively strong antibacterial activity against Klebsiella pneumoniae, Pseudomonas aeruginosa, Staphylococcus aureus, Escherichia coli, Enterococcus faecalis, Streptococcus pneumoniae, Enterobacter cloacae, and Vibrio cholerae. The antibacterial activities of these compounds against Klebsiella pneumoniae and Escherichia coli are stronger than those against Pseudomonas aeruginosa, Enterococcus faecalis, and Staphylococcus aureus. The in-vitro antibacterial activities of the S-ulifloxacin serial products against Pseudomonas aeruginosa are stronger than that of Levofloxacin, but close to that of Ciprofloxacin; their in-vitro antibacterial activities against Klebsiella pneumoniae, Escherichia coli, and Staphylococcus aureus are slightly better or close to those of Ciprofloxacin and Levofloxacin. If the concentrations of the ulifloxacin serial products are calculated based on their equivalents to NM394, the antibacterial activities of compounds 4, 5, and 6 against the above four bacteria are slightly stronger than those of compounds 7 and 8.

### Example 17

### Vascular Irritation Tests

Sixteen New Zealand rabbits were divided into 4 groups: a control group and three treatment groups for compounds 4, 5, and 6, respectively; each group had 4 rabbits. The rabbits of the treatment groups were vein-injected on their left ears with high doses of the test samples and on their right ears with low doses of the test samples. The rabbits of the control group were rein-injected on their both left and right ears with sodium chloride solution. The injections continued for 3 days, once a day. Two rabbits from each group were anatomized after 24 hours of each injection. The results indicate that 14 rabbit ears showed clear outline blood vessels, remained uniform thickness, and showed no significant changes. The pathological histology showed that rabbit ears had no toxicologically significant vascular changes. This indicates that compounds 4, 5, and 6 have no irritation to the in New Zealand rabbit ear vein and surrounding tissues.

### Example 18

### Toxicity Test

Compounds 4 and 5 were selected for the toxicity test. SD rats were administrated by intravenous injection of the compound samples and observed for toxicity reaction, severity, and reversibility of the damage to the main targeted organs. The dose without toxic reaction was thereby determined and the long term toxicity to SD rats was tested. One hundred forty SD rats were randomly divided into 7 groups depending on their body weight and sex; each group had 20 rats with 10 males and 10 females. Compounds 5 and 4 each had three dose groups, 10, 30, and 60 mg/kg·bw (calculation based on NM394); and there was a blank control group. The rats were administrated by intravenous injection, once a day for consecutive 4 weeks. The recovery was monitored for 2 weeks after treatment.

### Test Groups and Dose Settings Were:

As discussed above, the tests were divided into three dose groups and one blank control group; each group had 4 males and 4 females. The test groups and doses used are listed in Table 2.

TABLE 2
Test groups and doses

| | Dose | Number of Rats | |
|---|---|---|---|
| Group | (mg/kg · bw) | Female | Male |
| Blank Control Group | 0 | 10 | 10 |
| Compound 5, Low Dose Group | 10 | 10 | 10 |
| Compound 5, Medium Dose Group | 30 | 10 | 10 |
| Compound 5, High Dose Group | 60 | 10 | 10 |
| Compound 4, Low Dose Group | 10 | 10 | 10 |
| Compound 4, Medium Dose Group | 30 | 10 | 10 |
| Compound 4, High Dose Group | 60 | 10 | 10 |

Drug preparation: the drugs were dissolved with a saline solution to the desired concentration before administration.
Administration: intravenous injection.
Delivery period: 28 days continuously.
Observation and inspection:
Observation and inspection projects, targets and time: including the general state of observation (7), hematologic (16), biochemical blood test (18), urine (10) and pathological examination.

### Tested Drugs:

### Name: Compounds 4 and 5.

### Observation and Inspection Methods:

General observation: the appearance of signs, behavior, glands, respiratory and stool, daily observation and timely recording, discovering dead or dying animals, timely anatomical examination, food intake, and body weight once weekly.

Blood tests: abdominal aortic blood for hematology testing, in the 16 testing indicators, leukocyte classification using whole blood smear, Wright stain staining; prothrombin time (PT) with a 0.109 mmol/L of citrate Citrus medica sodium (with blood volume ratio of 1:9) anticoagulant, 3000 r/min centrifugal 10 min, taking with BE Thromotimer plasma coagulation analyzer (Germany) determination; other indicators to EDTA anticoagulant, using SWELAB AC920EO automated hematology analyzer (Sweden).

Biochemical blood tests: blood sampling method same as above, blood serum obtained by 3000 r/min centrifugal 10 min. In the 21 indicators, the Na+, K+, Cl⁻ were determined using EasyLyte Plus Na/K/Cl analyzer (USA), the other indexes of blood were determined using a Hitachi 7020 automatic biochemical analyzer (Japan) determination.

Urine tests: 16 h urine collection, using CLINITEK 100 urine analyzer.

Pathology: after anesthesia, the animal was killed by blood letting of the abdominal aorta. System anatomy and visual observation of changes in various organs were performed; and organ weights were determined. Tissue was fixed in 10% neutral formalin; plates were made by conventional paraffin producers, HE staining, and light microscopy.

Statistical analysis: In the different groups, the body weight, hematology, blood chemistry, urine measurements, organ weights, organ coefficient were calculated for the mean±standard deviation and t-test comparison between groups. Pathological abnormalities according to their incidence and severity were compared between groups.

### Test Results:

### Observation of General Conditions:

During the administration and recovery observation period, compared with the control group, the treatment group animals showed no abnormal changes in the body weight, food intake, appearance, behavior, gland secretion, respiratory conditions, and in stool.

### Hematology Tests:

After 4 weeks of the drug administration, the female rats of the low dose groups and the medium dose groups showed slight decrease in leukocyte and slight increase in lymphocytes. However, the differences were insignificant (P>0.05) compared with the rats of the control group, and there were no abnormal changes in other indicators. After 4 weeks of the drug administration, the female rats of the high dose groups showed slight decrease in leukocyte but significant increase in lymphocytes compared with the rats of the control group. After the recovery period, the Hematology indicators of all dose groups showed no abnormal changes.

### Blood Biochemical Tests:

The examinations for both after 4 weeks of the drug administration and after the recovery period showed no abnormal changes in the blood biochemical indicators (P>0.05), compared with the rats of the control group.

After 4 weeks of the drug administration, the male rats in the high dose groups showed significant increase in urea nitrogen and creatinine compared with the rats in the control group (P<0.05); other indicators showed no abnormal changes. After the recovery period, the rats in the high dose groups showed no abnormal changes in the blood biochemical indicators.

### Urea Tests

After 4 weeks of the drug administration, the male rats of all dose groups showed significant decrease (P<0.05) in the urea pH value compared with the control group; the male rats of the high dose group also showed a small amount of phosphate crystals in urine; and there were no other abnormal changes. After the recovery period, the rats of all dose groups showed no abnormal changes in the urea tests.

### Pathological Examination:

General pathology examination: compared with the control group, the male rats of the high dose groups, after 4 weeks of the drug administration, showed significant weight increase in both of the left and right kidneys (P<0.05) and significant increase in the adrenal weight coefficient (P<0.05); there were no other noticeable toxicological changes in the tissues, organs, organ weight, and organ coefficient.

Tissue microscopy: after 28 days of the continuous drug administration, two rats of the compound 5, high dose group showed mild cortical tubular dilatation, one rat showed kidney protein casts, and two rats (5 ♂, 7 ♂) showed crystals in renal tubules. After 28 days of the continuous drug administration, in the compound 4, high dose group, 3 rats showed mild cortical tubular dilatation, and one showed kidney protein casts. In the compound 4, high dose group, 1 rat showed mild interstitial inflammatory cell infiltration and 3 rats showed crystals in renal tubules. The microscopy of other organs and tissues in all dose groups showed no pathological changes of toxicological significance.

### Conclusion:

The above results indicate: for compounds 5 and 4, 28 days of continuous intravenous administration at a dose less than 30 mg/kg·bw (calculated based on NM394) caused no toxicologically significant changes to the rats; this dose was determined to be NOAEL (no observed adverse effect level) for the SD rats. When the continuous intravenous administration at a dose greater than 60 mg/kg·bw, the SD rats showed toxicity reaction; the targeted organ was the kidney; the blood-sensitive indicators were the blood creatinine and blood urea nitrogen; and the urine-sensitive indicators were phosphate crystals and the pH value. It can be hypothesized that compounds 5 and 4 in clinical application shall be closely monitored for changes in renal function and urine.

### INDUSTRIAL APPLICABILITY

The invention provides a fluorine-containing, optically active quinolone compound which can be used as an anti-infection medicine. The compound has defined structure and stable properties. Compared with ulifoxacin, the compound of the invention has improved water solubility. It is readily soluble in water. It is of relatively strong antibacterial activity, low toxicity to kidneys, and of no irritation to skin and muscle. Its NOAEL is 30 mg/kg, which is 10 times higher than that of ulifoxacin. Its side effects are low. It has increased clinical drug safety. It has a broad antimicrobial spectrum. Its antimicrobial activity is 1-3 times higher than that of the racemic ulifoxacin. In addition, the production process of the compound is simple, reasonable, and thus it has industrial applicability.

1.-7. (canceled)

8. A compound, comprising at least one fluorine, chosen from optically active quinolone compounds of formula 1: 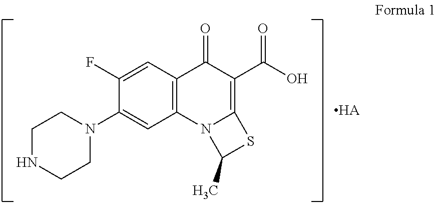
wherein HA is an organic or inorganic acid.

9. The compound of claim 8, wherein said organic acid is chosen from acetic acid, glycine, methylsulfonic acid, lactic acid, glutamic acid, mandelic acid, gluconic acid, aspartic acid, citric acid, succinic acid, fumaric acid, maleic acid, oxalic acid, lactose acid, and benzenesulfonic acid; and said inorganic acid is chosen from hydrochloric acid, hydrobromic acid, sulfuric acid, and phosphoric acid.

10. The compound of claim 9, wherein said organic acid is lactic acid.

11. The compound of claim 10, wherein the compound has the specific rotation [α]_{D}²⁰ from −112.5° to −118.2° and IR absorptions at 1698 cm⁻¹, 1629 cm⁻¹, 1605 cm⁻¹, 1501 cm⁻¹, 1396 cm⁻¹, and 1257 cm⁻¹.

12. The compound of claim 9, wherein said organic acid is methylsulfonic acid.

13. The compound of claim 12, wherein the compound has the specific rotation [α]_{D}²⁰ from −106.6° to −112.6° and IR absorptions at 1707 cm⁻¹, 1629 cm⁻¹, 1602 cm⁻¹, and 1501 cm⁻¹.

14. The compound of claim 9, wherein said organic acid is gluconic acid.

15. The compound of claim 14, wherein the compound has IR absorptions at 1695 cm⁻¹, 1629 cm⁻¹, 1601 cm⁻¹, and 1503 cm⁻¹.

16. The compound of claim 9, wherein said organic acid is glutamic acid.

17. The compound of claim 16, wherein the compound has IR absorptions at 1628 cm⁻¹, 1603 cm⁻¹, 1499 cm⁻¹, 1457 cm⁻¹, 1397 cm⁻¹, and 1257 cm⁻¹.

18. The compound of claim 9, wherein said organic acid is aspartic acid.

19. The compound of claim 18, wherein the compound has IR absorptions at 1695 cm⁻¹, 1628 cm⁻¹, 1602 cm⁻¹, and 1499 cm⁻¹.

20. A pharmaceutical composition, comprising the compound of claim 8 and at least one pharmaceutically acceptable carrier.

21. The pharmaceutical composition of claim 20, wherein the pharmaceutical composition is in a form chosen from oral dosage forms and non-gastrointestinal delivery forms.

22. The pharmaceutical composition of claim 21, wherein the oral dosage forms are chosen from tablets, capsules, powders, and granular agents of solid.

23. The pharmaceutical composition of claim 21, wherein the non-gastrointestinal delivery forms are chosen from injections.

24. The pharmaceutical composition of claim 22, wherein the capsules are chosen from sustained-release and controlled-release formulations.

25. A pharmaceutical composition, comprising the compound of claim 9 and at least one pharmaceutically acceptable carrier.

26. A method of treating an infectious disease, comprising administering to a patient in need thereof the pharmaceutical composition of claim 20.

27. A method of treating an infectious disease, comprising administering to a patient in need thereof the pharmaceutical composition of claim 25.
